(19) United States
(12) Patent Application Publication
(10) Pub. No.: US 2021/0352925 A1
(43) Pub. Date: Nov. 18, 2021

(54) Fermented formula with non digestible oligosaccharides for sleep improvement

(71) Applicant: N.V. Nutricia, Zoetermeer (NL)

(72) Inventors: Houkje Bouritius, Utrecht (NL); Kelly Ann Mulder, Utrecht (NL); Marieke Abrahamse-Berkeveld, Utrecht (NL)

(73) Assignee: N.V. Nutricia, Zoetermeer (NL)

(21) Appl. No.: 17/283,269

(22) PCT Filed: Feb. 4, 2020
(86) PCT No.: PCT/EP2020/052707

§ 371 Date: Apr. 7, 2021

(30) Foreign Application Priority Data
Feb. 4, 2019 (EP) 19155299.1

(51) Int. Cl. A23C 9/123; A23C 9/20; A23L 33/135; A23L 33/21; A23L 33/00

(52) U.S. Cl. CPC A23C 9/1234; A23C 9/203; A23L 33/40; A23L 33/21; A23L 33/135

(57) ABSTRACT

The present invention concerns improving sleep in infants by administering nutrition that is at least partly fermented by lactic acid producing bacteria and comprises non-digestible oligosaccharides.

### FIELD OF THE INVENTION

The present invention relates to the field of nutrition for infants for improving sleep

### BACKGROUND OF THE INVENTION

Breastfeeding is the preferred source of nutrition for infants and has been proven to provide a range of short-term and long-term benefits for the child's nervous, immune, metabolic and gastrointestinal system. Because breastfeeding may not always be possible, breast milk substitutes should aim to provide nutritional and functional properties as close as possible to those of human milk.

Over the past decades, several studies have indicated that partly fermented infant milk formulae comprising non-digestible oligosaccharides galacto-oligosaccharides and long chain fructo-oligosaccharides have a beneficial effect on gut comfort and reduce crying and colics (WO 2015/065194), increase the production of intestinal secretory IgA (WO 2017/114900), and improve the growth trajectory and eating behavior (WO 2017/194615, WO 2017/194607) in infants. Such formulae are produced using fermentation processes using food-grade microorganisms to generate bioactive compounds, which are also known as postbiotics.

Sleep patterns develops or matures rapidly during the first few years of life and is a highly dynamic process. At birth, infants lack an established circadian rhythm and hence sleep across multiple intervals throughout the day and night in short bouts. This is also due to infants' feeding needs, as newborns have a small stomach and must wake every few hours to eat. At about 10-12 weeks of age, the first signs of a circadian rhythm begin to develop, marked by an increased ease of sleeping through the night. The change in total sleep duration over 24 hours continues and decreases from 16 to 17 hours in newborns, to 14-15 hours at 16 weeks of age, and 13-14 hours by 6 months of age. The National Sleep Foundation (NSF) recommends a daily sleep duration of 14-17 hours/day from birth to 3 months, 12-15 hours/day from 4 to 11 months, 11-14 hours/day for infants aged 1-2 years, and 10-13 hours/day for preschoolers aged 3-5 years.

Scientific literature supports a critical and positive role of infant sleep in cognition and physical growth (Tham et al, 2017 Nature and Science of Sleep, 9: 135-149). There is a positive association between sleep, memory, language, executive function, and overall cognitive development in developing infants and young children. Additionally, infant sleep has a positive role in physical growth. Furthermore, infant sleep disturbances inevitably lead to parental sleep disturbance and stress which may result in inadequate child-parental interaction. Hence a good sleep efficiency and the development or maturation of a normal sleep pattern in infants is highly desired and beneficial.

WO 2006/034955 discloses an infant nutritional kit which comprises a wakefulness stimulating formula and a sleep stimulating formula, wherein the two formulae differ in tryptophan, nucleotides and medium chain triglycerides.

WO 2010/060722 discloses the use of a probiotic bacterial strain in the manufacture of a medicament or therapeutic nutritional composition for improving maturation of sleep patterns in infants, young children or young animals and/or for reducing sleep disturbances and/or improving sleep patterns in humans or animals at any age. A rat model was used, employing prenatal stress to disturb sleep behavior.

### SUMMARY OF THE INVENTION

In a randomized, controlled, double-blinded, clinical trial the effect of a partly fermented infant formula comprising non-digestible oligosaccharides was evaluated in healthy, term infants on the incidence of GI (related) symptoms, sleep behavior, as well as growth adequacy and safety. As a control formula a non-fermented formula without non-digestible oligosaccharides was used. Both experimental and control formulae were safe, well tolerated and supported adequate growth. Surprisingly in infants receiving the experimental formula the number of sleep episodes per 24 h was significantly reduced compared to infants receiving control formula, when reaching an age above 3 months (13 weeks). The total sleep time (h per 24 h), however, was not affected. This shows that sleep was improved, e.g. efficiency of sleep was improved or maturation of sleep was improved.

### DETAILED DESCRIPTION OF THE INVENTION

Thus the present invention concerns a method for improving sleep behavior and/or improving sleep pattern in an infant, comprising administering a nutritional composition that is at least partly fermented by lactic acid producing bacteria and comprises non-digestible oligosaccharides to the infant.

In one embodiment, improving sleep behavior and/or improving sleep pattern comprises improving sleep efficiency, decreasing sleep frequency, decreasing wake frequency and/or increasing sleep episode duration in the infant. Preferably improving sleep behavior and/or improving sleep pattern occurs in the infant above 3 months of age.

In one embodiment, improving sleep behavior and/or improving sleep pattern comprises improving the development of sleep pattern and/or improving maturation of sleep pattern in the infant. Preferably development of sleep pattern and/or maturation of sleep pattern occurs in the infant under 3 months of age.

For some jurisdictions the invention may be worded as the use of a nutritional composition that is at least partly fermented by lactic acid producing bacteria and comprises non-digestible oligosaccharides for improving sleep behavior and/or improving sleep pattern in an infant.

In one embodiment, the use for improving sleep behavior and/or improving sleep pattern in an infant comprises improving sleep efficiency, decreasing sleep frequency, decreasing wake frequency and/or increasing sleep episode duration in the infant.

In one embodiment, the use for improving sleep behavior and/or improving sleep pattern in an infant comprises improving the development of sleep pattern and/or improving maturation of sleep pattern in the infant.

### Fermented Composition

The nutritional composition in the methods or uses according to the present invention, hereafter also referred to as the present nutritional composition, or nutritional composition of the invention or final nutritional composition, is at least party fermented. A partly fermented nutritional composition comprises at least for a part a composition that was fermented by lactic acid producing bacteria. It was shown that the presence of fermented composition in the final nutritional composition results, upon administration, in an improvement of sleep.

The fermentation preferably takes place during the production process of the nutritional composition. Preferably, the nutritional composition does not contain significant amounts of viable bacteria in the final product, and this can be achieved by heat inactivation after fermentation or inactivation by other means. Preferably the fermented composition is a milk-derived product, which is a milk substrate that is fermented by lactic acid producing bacteria, wherein the milk substrate comprises at least one selected from the group consisting of milk, whey, whey protein, whey protein hydrolysate, casein, casein hydrolysate or mixtures thereof. Suitably, nutritional compositions comprising fermented compositions and non-digestible oligosaccharide and their way of producing them are described in WO 2009/151330, WO 2009/151331 and WO 2013/187764.

The fermented composition preferably comprises bacterial cell fragments like glycoproteins, glycolipids, peptidoglycan, lipoteichoic acid (LTA), lipoproteins, nucleotides, and/or capsular polysaccharides. It is of advantage to use the fermented composition comprising inactivated bacteria and/or cell fragments directly as a part of the final nutritional product, since this will result in a higher concentration of bacterial cell fragments. When commercial preparations of lactic acid producing bacteria are used, these are usually washed and material is separated from the aqueous growth medium comprising the bacterial cell fragments, thereby reducing or eliminating the presence of bacterial cell fragments. Furthermore, upon fermentation and/or other interactions of lactic acid producing bacteria with the milk substrate, additional bio-active compounds can be formed, such as short chain fatty acids, bioactive peptides and/or oligosaccharides, and other metabolites, which may also result in an intestinal microbiota-function more similar to the intestinal microbiota-function of breastfed infants. Such bioactive compounds that that are produced during fermentation by lactic acid producing bacteria may also be referred to as post-biotics. A composition comprising such post-biotics is thought to be advantageously closer to breast milk, as breast milk is not a clean synthetic formula, but contains metabolites, bacterial cells, cell fragments and the like. Therefore the fermented composition, in particular fermented milk-derived product, is believed to have an improved effect compared to non-fermented milk-derived product without or with merely lactic acid producing bacteria on the sleep function in infants.

Preferably the final nutritional composition comprises 5 to 97.5 wt % of the fermented composition based on dry weight, more preferably 10 to 90 wt %, more preferably 20 to 80 wt %, even more preferably 25 to 60 wt %. As a way to specify that the final nutritional composition comprises at least partly a fermented composition, and to specify the extent of fermentation, the level of the sum of lactic acid and lactate in the final nutritional composition can be taken, as this is the metabolic end product produced by the lactic acid producing bacteria upon fermentation. The present final nutritional composition preferably comprises 0.1 to 1.5 wt % of the sum of lactic acid and lactate based on dry weight of the composition, more preferably 0.1 to 1.0 wt %, even more preferably 0.2 to 0.5 wt %. Preferably at least 50 wt %, even more preferably at least 90 wt %, of the sum of lactic acid and lactate is in the form of the L(+)-isomer. Thus in one embodiment the sum of L(+)-lactic acid and L(+)-lactate is more than 50 wt %, more preferably more than 90 wt %, based on the sum of total lactic acid and lactate. Herein L(+)-lactate and L(+)-lactic acid is also referred to as L-lactate and L-lactic acid.

### Lactic Acid Producing Bacteria Used for Producing the Fermented Ingredient

Lactic acid producing bacteria used for preparing the fermented ingredient, in particular for fermentation of the milk substrate are preferably provided as a mono- or mixed culture. Lactic acid producing bacteria consists of the genera Bifidobacterium, Lactobacillus, Carnobacterium, Enterococcus, Lactococcus, Leuconostoc, Oenococcus, Pediococcus, Streptococcus, Tetragenococcus, Vagococcus and Weissella. Preferably the lactic acid producing bacteria used for fermentation comprises bacteria of the genus Bifidobacterium and/or Streptococcus.

Preferably the Streptococcus is a strain of S. thermophilus. Selection of a suitable strain of S. thermophilus is described in example 2 of EP 778885 and in example 1 of FR 2723960. In a further preferred embodiment according to the present invention, the nutritional composition comprises 10²-10⁵ cfu living bacteria of S. thermophilus, per g dry weight of the final nutritional composition, preferably the final nutritional composition comprises 10³-10⁴ living bacteria of S. thermophilus per g dry weight.

Preferred strains of S. thermophilus to prepare the fermented ingredient for the purpose of the present invention have been deposited by Compagnie Gervais Danone at the Collection Nationale de Cultures de Microorganismes (CNCM) run by the Institut Pasteur, 25 rue du Docteur Roux, Paris, France on 23 Aug. 1995 under the accession number 1-1620 and on 25 Aug. 1994 under the accession number 1-1470. Other S. thermophilus strains are commercially available.

Bifidobacteria are Gram-positive, anaerobic, rod-shaped bacteria. Preferred Bifidobacterium species to prepare the fermented ingredient for the purpose of the present invention preferably have at least 95% identity of the 16 S rRNA sequence when compared to the type strain of the respective Bifidobacterium species, more preferably at least 97% identity as defined in handbooks on this subject for instance Sambrook, J., Fritsch, E. F., and Maniatis, T. (1989), Molecular Cloning, A Laboratory Manual, 2nd ed., Cold Spring Harbor (N.Y.) Laboratory Press. The bifidobacteria preferably used are also described by Scardovi, V. Genus Bifidobacterium. p. 1418-p. 1434. In: Bergey's manual of systematic Bacteriology. Vol. 2. Sneath, P. H. A., N. S. Mair, M. E. Sharpe and J. G. Holt (ed.). Baltimore: Williams & Wilkins. 1986. 635 p. Preferably the lactic acid producing bacteria used for fermentation comprises or is at least one Bifidobacterium selected from the group consisting of B. breve, B. infantis, B. bifidum, B. catenulatum, B. adolescentis, B. thermophilum, B. gallicum, B. animalis or lactis, B. angulatum, B. pseudocatenulatum, B. thermacidophilum and B. longum more preferably B. breve, B. infantis, B. bifidum, B. catenulatum, B. longum, more preferably B. longum and B. breve, even more preferably B. breve, more preferably B. breve selected from the group consisting of B. breve Bb-03 (Rhodia/Danisco), B. breve M-16V (Morinaga), B. breve R0070 (Institute Rosell, Lallemand), B. breve BRO3 (Probiotical), B. breve BR92 (Cell Biotech) DSM 20091, LMG 11613 and B. breve 1-2219 deposited at the CNCM, Paris France. Most preferably, the B. breve is B. breve M-16V (Morinaga) or B. breve 1-2219, even more preferably B. breve 1-2219.

Most preferably the nutritional composition of the invention comprises fermented composition that is fermented by lactic acid producing bacteria comprising both B. breve and S. thermophilus. In one embodiment the fermentation by lactic acid producing bacteria is fermentation by Streptococcus thermophilus and Bifidobacterium breve. In one embodiment, the final nutritional composition comprises fermented composition wherein the lactic acid producing bacteria are inactivated after fermentation. Thus in one embodiment, the final nutritional composition comprises fermented composition that comprises inactivated lactic acid producing bacteria, preferably the final nutritional composition comprises fermented composition that comprises inactivated Bifidobacteria and/or inactivated Streptococcus, preferably the final nutritional composition comprises fermented composition that comprises inactivated B. breve and/or inactivated S. thermophilus, preferably the final nutritional composition comprises fermented composition that comprises inactivated Bifidobacteria and inactivated Streptococcus, preferably the final nutritional composition comprises fermented composition that comprises inactivated B. breve and/or inactivated S. thermophilus, preferably inactivated B. breve and inactivated S. thermophilus. In other words, the present nutritional composition comprises lactic acid producing bacteria, preferably inactivated lactic acid producing bacteria. Preferably the present nutritional composition comprises lactic acid producing bacteria that are selected from the group consisting of Bifidobacterium and Streptococcus, preferably are selected both, preferably the lactic acid producing bacteria are selected from the group consisting of Bifidobacterium breve and Streptococcus thermophilus, preferably are selected both. Preferably the present nutritional composition comprises inactivated lactic acid producing bacteria that are selected from the group consisting of Bifidobacterium and Streptococcus, preferably are selected both, preferably the inactivated lactic acid producing bacteria are selected from the group consisting of Bifidobacterium breve and Streptococcus thermophilus, preferably are selected both.

Preferably the fermented composition is not fermented by Lactobacillus bulgaricus. L. bulgaricus fermented products are considered not suitable for infants, since in young infants the specific dehydrogenase that converts D-lactate to pyruvate is far less active than the dehydrogenase which converts L-lactate.

Preferably the nutritional composition of the invention comprises inactivated lactic acid producing bacteria and/or bacterial fragments derived from lactic acid producing bacteria being the equivalent of more than 1×10⁴ cfu lactic acid producing bacteria per g based on dry weight of the final composition, more preferably 1×10⁵ cfu, even more preferably 1×10⁶ cfu. Preferably the inactivated bacteria or bacterial fragments are the equivalent of less than 1×10¹³ cfu lactic acid producing bacteria per g based on dry weight of the final composition, more preferably 1×10¹¹ cfu, even more preferably 1×10¹⁰ cfu. The correlation of inactivated lactic acid bacteria and the equivalence with cfu can be determined by molecular techniques, known in the art, or by checking the production process.

### Process of Fermentation

Preferably the fermented composition is a milk-derived product, which is a milk substrate that is fermented by lactic acid producing bacteria, and said milk substrate comprising at least one selected from the group consisting of milk, whey, whey protein, whey protein hydrolysate, casein, casein hydrolysate or mixtures thereof. The milk derived product or milk substrate to be fermented is suitably present in an aqueous medium. The milk substrate to be fermented comprises at least one selected from the group consisting of milk, whey, whey protein, whey protein hydrolysate, casein, casein hydrolysate or mixtures thereof. Milk can be whole milk, semi-skimmed milk and/or skimmed milk. Preferably the milk substrate to be fermented comprises skimmed milk. Whey can be sweet whey, and/or acid whey. Preferably the whey is present in a concentration of 3 to 80 g dry weight per l aqueous medium containing milk substrate, more preferably 40 to 60 g per l. Preferably whey protein hydrolysate is present in 2 to 80 g dry weight per l aqueous medium containing milk substrate, more preferably 5 to 15 g/l. Preferably lactose is present in 5 to 50 g dry weight per l aqueous substrate, more preferably 1 to 30 g/l. Preferably the aqueous medium containing milk substrate comprises buffer salts in order to keep the pH within a desired range. Preferably sodium or potassium dihydrogen phosphate is used as buffer salt, preferably in 0.5 to 5 g/l, more preferably 1.5 to 3 g per l. Preferably the aqueous medium containing milk substrate comprises cysteine in amount of 0.1 to 0.5 g per l aqueous substrate, more preferably 0.2 to 0.4 g/l. The presence of cysteine results in low redox potential of the substrate which is advantageous for activity of lactic acid producing bacteria, particularly bifidobacteria. Preferably the aqueous medium containing milk substrate comprises yeast extract in an amount of 0.5 to 5 g/l aqueous medium containing milk substrate, more preferably 1.5 to 3 g/l. Yeast extract is a rich source of enzyme co-factors and growth factors for lactic acid producing bacteria. The presence of yeast extract will enhance the fermentation by lactic acid producing bacteria.

Suitably the milk substrate, in particular the aqueous medium containing milk substrate, is pasteurised before the fermentation step, in order to eliminate the presence of unwanted living bacteria. Suitably the product is pasteurised after fermentation, in order to inactivate enzymes. Suitably the enzyme inactivation takes place at 75° C. for 3 min. Suitably the aqueous medium containing milk substrate is homogenised before and/or the milk-derived product is homogenised after the fermentation. Homogenisation results in a more stable substrate and/or fermented product, especially in the presence of fat.

The inoculation density is preferably between 1×10² to 5×10¹⁰, preferably between 1×10⁴ to 5×10⁹ cfu lactic acid producing bacteria/ml aqueous medium containing milk substrate, more preferably between 1×10⁷ to 1×10⁹ cfu lactic acid producing bacteria/ml aqueous medium containing milk substrate. The final bacteria density after fermentation is preferably between 1×10³ to 1×10¹⁰, more preferably between 1×10⁴ to 1×10⁹ cfu/ml aqueous medium containing milk substrate.

The fermentation is preferably performed at a temperature of approximately 20° C. to 50° C., more preferably 30° C. to 45° C., even more preferably approximately 37° C. to 42° C. The optimum temperature for growth and/or activity for lactic acid producing bacteria, more particularly lactobacilli and/or bifidobacteria is between 37° C. and 42° C.

The incubation is preferably performed at a pH of 4 to 8, more preferably 6 to 7.5. This pH does not induce protein precipitation and/or an adverse taste, while at the same time lactic acid producing bacteria such as lactobacilli and/or bifidobacteria are able to ferment the milk substrate.

The incubation time preferably ranges from 10 minutes to 48 h, preferably from 2 h to 24 h, more preferably from 4 h to 12 h. A sufficient long time enables fermentation and the concomitant production of immunogenic cell fragments such as glycoproteins, glycolipids, peptidoglycan, lipoteichoic acid (LTA), flagellae, lipoproteins, DNA and/or capsular polysaccharides and metabolites (postbiotics) to take place at a sufficient or higher extent, whereas the incubation time needs not be unnecessarily long for economic reasons.

Preferably, a milk derived product or milk substrate, preferably skimmed milk, is pasteurized, cooled and fermented with one or more lactic acid producing strains, preferably a strain of S. thermophilus, to a certain degree of acidity, upon which the fermented product is cooled and stored. Preferably a second milk-derived product is prepared in a similar way using one or more Bifidobacterium species for fermentation. Subsequently, the two fermented products are preferably mixed together and mixed with other components making up an infant formula, except the fat component. Preferably, the mixture is preheated, and subsequently fat is added in-line, homogenized, pasteurized and dried. Alternatively the fermentation takes place having both Bifidobacterium, preferably B. breve, and S. thermophilus in the fermentation tank.

Procedures to prepare fermented composition suitable for the purpose of the present invention are known per se. EP 778885, which is incorporated herein by reference, discloses in particular in example 7 a suitable process for preparing a fermented ingredient. FR 2723960, which is incorporated herein by reference, discloses in particular in example 6 a suitable process for preparing a fermented ingredient. Briefly, a milk substrate, preferably pasteurised, containing lactose and optionally further macronutrients such as fats, preferably vegetable fats, casein, whey protein, vitamins and/or minerals etc. is concentrated, e.g. to between 15 to 50% dry matter and then inoculated with S. thermophilus, for example with 5% of a culture containing 10⁶ to 10¹⁰ bacteria per ml. Preferably this milk substrate comprises milk protein peptides. Temperature and duration of fermentation are as mentioned above. Suitably after fermentation the fermented ingredient may be pasteurised or sterilized and for example spray dried or lyophilised to provide a form suitable to be formulated in the end product.

A preferred method for preparing the fermented composition to be used in the nutritional composition of invention is disclosed in WO 01/01785, more particular in examples 1 and 2. A preferred method for preparing the fermented composition to be used in the nutritional composition of invention is described in WO 2004/093899, more particularly in example 1.

Living cells of lactic acid producing bacteria in the fermented composition are after fermentation preferably eliminated, for example by inactivation and/or physical removal. The cells are preferably inactivated. Preferably the lactic acid producing bacteria are heat killed after fermentation of the milk substrate. Preferable ways of heat killing are (flash) pasteurization, sterilization, ultra-high temperature treatment, high temperature/short time heat treatment, and/or spray drying at temperatures bacteria do not survive. Cell fragments are preferably obtained by heat treatment. With this heat treatment preferably at least 90% of living microorganisms are inactivated, more preferably at least 95%, even more preferably at least 99%. Preferably the fermented nutritional composition comprises less than 1×10⁵ colony forming units (cfu) living lactic acid bacteria per g dry weight. The heat treatment preferably is performed at a temperature ranging from 70 to 180° C., preferably from 80 to 150° C., preferably for about 3 minutes to 2 hours, preferably in the range of 80 to 140° C. for 5 minutes to 40 minutes. Inactivation of the lactic acid bacteria advantageously results in less post acidification and a safer product. This is especially advantageous when the nutritional composition is to be administered to infants. Suitably after fermentation the fermented ingredient may be pasteurised or sterilized and for example spray dried or lyophilised to provide a form suitable to be formulated in the end product.

### Non-Digestible Oligosaccharides

The present nutritional composition comprises non-digestible oligosaccharides and preferably comprises at least two different non-digestible oligosaccharides, in particular two different sources of non-digestible oligosaccharides. The presence of non-digestible oligosaccharides is needed to improve the sleep functioning in infants. The presence of both the non-digestible oligosaccharides and the at least partly fermented composition, in particular the milk-derived product obtained by fermentation with lactic acid producing bacteria, is needed to improve sleep in infants.

The term “oligosaccharides” as used herein refers to saccharides with a degree of polymerization (DP) of 2 to 250, preferably a DP 2 to 100, more preferably 2 to 60, even more preferably 2 to 10. If oligosaccharide with a DP of 2 to 100 is included in the present nutritional composition, this results in compositions that may contain oligosaccharides with a DP of 2 to 5, a DP of 50 to 70 and a DP of 7 to 60. The term “non-digestible oligosaccharides” as used in the present invention refers to oligosaccharides which are not digested in the intestine by the action of acids or digestive enzymes present in the human upper digestive tract, e.g. small intestine and stomach, but which are preferably fermented by the human intestinal microbiota. For example, sucrose, lactose, maltose and maltodextrins are considered digestible.

Preferably the present non-digestible oligosaccharides are soluble. The term “soluble” as used herein, when having reference to a polysaccharides, fibres or oligosaccharides, means that the substance is at least soluble according to the method described by L. Prosky et al., J. Assoc. Off. Anal. Chem. 71, 1017-1023 (1988).

The non-digestible oligosaccharides included in the present nutritional compositions in the methods or uses according to the present invention preferably include a mixture of different non-digestible oligosaccharides. The non-digestible oligosaccharides are preferably selected from the group consisting of fructo-oligosaccharides, such as inulin, non-digestible dextrins, galacto-oligosaccharides, such as transgalacto-oligosaccharides, xylo-oligosaccharides, arabino-oligosaccharides, arabinogalacto-oligosaccharides, gluco-oligosaccharides, gentio-oligosaccharides, glucomanno-oligosaccharides, galactomanno-oligosaccharides, mannan-oligosaccharides, isomalto-oligosaccharides, nigero-oligosaccharides, glucomanno-oligosaccharides, chito-oligosaccharides, soy oligosaccharides, uronic acid oligosaccharides, fuco-oligosaccharides, sialyloligosaccharides and mixtures thereof. Such oligosaccharides share many biochemical properties and have similar functional benefits including improving the intestinal microbiota-function. Yet is understood that some non-digestible oligosaccharides and preferably some mixtures have an even further improved effect. Therefore more preferably the non-digestible oligosaccharides are selected from the group consisting of fructo-oligosaccharides, such as inulin, and galacto-oligosaccharides, such as betagalacto-oligosaccharides, and mixtures thereof, even more preferably betagalacto-oligosaccharides and/or inulin, most preferably betagalacto-oligosaccharides. In one embodiment in the nutritional composition according to the present invention, the non-digestible oligosaccharides are selected from the group consisting of galacto-oligosaccharides, fructo-oligosaccharides and mixtures of thereof, more preferably betagalacto-oligosaccharides, fructo-oligosaccharides and mixtures thereof.

The non-digestible oligosaccharides are preferably selected from the group consisting of betagalacto-oligosaccharides, alphagalacto-oligosaccharides, and galactan. According to a more preferred embodiment non-digestible oligosaccharides are betagalacto-oligosaccharides. Preferably the non-digestible oligosaccharides comprise galacto-oligosaccharides with beta(1,4), beta(1,3) and/or beta(1,6) glycosidic bonds and a terminal glucose. Transgalacto-oligosaccharides is for example available under the trade name Vivinal®GOS (Domo FrieslandCampina Ingredients), Bi2muno (Clasado), Cup-oligo (Nissin Sugar) and Oligomate55 (Yakult).

The non-digestible oligosaccharides preferably comprise fructo-oligosaccharides. Fructo-oligosaccharides may in other context have names like fructopolysaccharides, oligofructose, polyfructose, polyfructan, inulin, levan and fructan and may refer to oligosaccharides comprising beta-linked fructose units, which are preferably linked by beta(2,1) and/or beta(2,6) glycosidic linkages, and a preferable DP between 2 and 200. Preferably, the fructo-oligosaccharides contain a terminal beta(2,1) glycosidic linked glucose. Preferably, the fructo-oligosaccharides contain at least 7 beta-linked fructose units. In a further preferred embodiment inulin is used. Inulin is a type of fructo-oligosaccharides wherein at least 75% of the glycosidic linkages are beta(2,1) linkages. Typically, inulin has an average chain length between 8 and 60 monosaccharide units. A suitable fructo-oligosaccharides for use in the compositions of the present invention is commercially available under the trade name Raftiline®HP (Orafti). Other suitable sources are Raftilose (Orafti), Fibrulose and Fibruline (Cosucra) and Frutafit and Frutalose (Sensus).

Preferably the present nutritional composition comprises a mixture of galacto-oligosaccharides and fructo-oligosaccharides. Preferably the mixture of galacto-oligosaccharides and fructo-oligosaccharides is present in a weight ratio of from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1. This weight ratio is particularly advantageous when the galacto-oligosaccharides have a low average DP and fructo-oligosaccharides has a relatively high DP. Most preferred is a mixture of galacto-oligosaccharides with an average DP below 10, preferably below 6, and fructo-oligosaccharides with an average DP above 7, preferably above 11, even more preferably above 20.

Preferably the present nutritional composition comprises a mixture of short chain fructo-oligosaccharides and long chain fructo-oligosaccharides. Preferably the mixture of short chain fructo-oligosaccharides and long chain fructo-oligosaccharides is present in a weight ratio of from 1/99 to 99/1, more preferably from 1/19 to 19/1, even more preferably from 1/10 to 19/1, more preferably from 1/5 to 15/1, more preferably from 1/1 to 10/1. Preferred is a mixture of short chain fructo-oligosaccharides with an average DP below 10, preferably below 6 and a fructo-oligosaccharides with an average DP above 7, preferably above 11, even more preferably above 20.

Preferably the present nutritional composition comprises a mixture of short chain fructo-oligosaccharides and short chain galacto-oligosaccharides. Preferably the mixture of short chain fructo-oligosaccharides and short chain galacto-oligosaccharides is present in a weight ratio of from 1/99 to 99/1, more preferably from 1/19 to 19/1, even more preferably from 1/10 to 19/1, more preferably from 1/5 to 15/1, more preferably from 1/1 to 10/1. Preferred is a mixture of short chain fructo-oligosaccharides and galacto-oligosaccharides with an average DP below 10, preferably below 6.

The present nutritional composition preferably comprises 2.5 to 20 wt % total non-digestible oligosaccharides, more preferably 2.5 to 15 wt %, even more preferably 3.0 to 10 wt %, most preferably 5.0 to 7.5 wt %, based on dry weight of the nutritional composition. Based on 100 ml the present nutritional composition preferably comprises 0.35 to 2.5 wt % total non-digestible oligosaccharides, more preferably 0.35 to 2.0 wt %, even more preferably 0.4 to 1.5 wt %, based on 100 ml of the nutritional composition. A lower amount of non-digestible oligosaccharides will be less effective in improving the sleep, whereas a too high amount will result in side-effects of bloating and abdominal discomfort.

### Nutritional Composition

The nutritional composition used according to the present invention is preferably for enteral administration, more preferably for oral administration.

The present nutritional composition is preferably an infant formula or follow on formula. More preferably the nutritional composition is an infant formula. The present nutritional composition can be advantageously applied as a complete nutrition for infants. Preferably the present nutritional composition is an infant formula. An infant formula is defined as a formula for use in infants and can for example be a starter formula, intended for infants of 0 to 6 or 0 to 4 months of age. A follow on formula is intended for infants of 4 or 6 months to 12 months of age. At this age infants start weaning on other food. The present composition preferably comprises a lipid component, protein component and carbohydrate component and is preferably administered in liquid form. The present nutritional composition may also be in the form of a dry food, preferably in the form of a powder which is accompanied with instructions as to mix said dry food, preferably powder, with a suitable liquid, preferably water. The nutritional composition used according to the invention preferably comprises other fractions, such as vitamins, minerals, trace elements and other micronutrients in order to make it a complete nutritional composition. Preferably infant formulae comprise vitamins, minerals, trace elements and other micronutrients according to international directives.

The present nutritional composition preferably comprises lipid, protein and digestible carbohydrate wherein the lipid provides 5 to 50% of the total calories, the protein provides 5 to 50% of the total calories, and the digestible carbohydrate provides 15 to 90% of the total calories. Preferably, in the present nutritional composition the lipid provides 35 to 50% of the total calories, the protein provides 7.0 to 12.5% of the total calories, and the digestible carbohydrate provides 40 to 55% of the total calories. For calculation of the % of total calories for the protein, the total of energy provided by proteins, peptides and amino acids needs to be taken into account. Preferably the lipid provides 3 to 7 g lipid per 100 kcal, preferably 4 to 6 g per 100 kcal, the protein provides 1.6 to 4 g per 100 kcal, preferably 1.7 to 2.5 g per 100 kcal and the digestible carbohydrate provides 5 to 20 g per 100 kcal, preferably 8 to 15 g per 100 kcal of the nutritional composition. Preferably the present nutritional composition comprises lipid providing 4 to 6 g per 100 kcal, protein providing 1.6 to 2.0 g per 100 kcal, more preferably 1.7 to 1.9 g per 100 kcal and digestible carbohydrate providing 8 to 15 g per 100 kcal of the nutritional composition. In one embodiment, the lipid provides 3 to 7 g lipid per 100 kcal, preferably 4 to 6 g per 100 kcal, the protein provides 1.6 to 2.1 g per 100 kcal, preferably 1.6 to 2.0 g per 100 kcal and the digestible carbohydrate provides 5 to 20 g per 100 kcal, preferably 8 to 15 g per 100 kcal of the nutritional composition and wherein preferably the digestible carbohydrate component comprises at least 60 wt % lactose based on total digestible carbohydrate, more preferably at least 75 wt %, even more preferably at least 90 wt % lactose based on total digestible carbohydrate. The amount of total calories is determined by the sum of calories derived from protein, lipids, digestible carbohydrates and non-digestible oligosaccharide.

The present nutritional composition preferably comprises a digestible carbohydrate component. Preferred digestible carbohydrate components are lactose, glucose, sucrose, fructose, galactose, maltose, starch and maltodextrin. Lactose is the main digestible carbohydrate present in human milk. The present nutritional composition preferably comprises lactose. As the present nutritional composition comprises a fermented composition that is obtained by fermentation by lactic acid producing bacteria, the amount of lactose is reduced compared to its source due to the fermentation whereby lactose is converted into lactate and/or lactic acid. Therefore in the preparation of the present nutritional composition lactose is preferably added. Preferably the present nutritional composition does not comprise high amounts of carbohydrates other than lactose. Compared to digestible carbohydrates such as maltodextrin, sucrose, glucose, maltose and other digestible carbohydrates with a high glycemic index, lactose has a lower glycemic index and is therefore preferred. The present nutritional composition preferably comprises digestible carbohydrate, wherein at least 35 wt %, more preferably at least 50 wt %, more preferably at least 60 wt %, more preferably at least 75 wt %, even more preferably at least 90 wt %, most preferably at least 95 wt % of the digestible carbohydrate is lactose. Based on dry weight the present nutritional composition preferably comprises at least 25 wt % lactose, preferably at least 40 wt %, more preferably at least 50 wt % lactose.

The present nutritional composition preferably comprises at least one lipid selected from the group consisting of animal lipid (excluding human lipids) and vegetable lipids. Preferably the present composition comprises a combination of vegetable lipids and at least one oil selected from the group consisting of fish oil, animal oil, algae oil, fungal oil, and bacterial oil. The lipid of the present nutritional composition preferably provides 3 to 7 g per 100 kcal of the nutritional composition, preferably the lipid provides 4 to 6 g per 100 kcal. When in liquid form, e.g. as a ready-to-feed liquid, the nutritional composition preferably comprises 2.1 to 6.5 g lipid per 100 ml, more preferably 3.0 to 4.0 g per 100 ml. Based on dry weight the present nutritional composition preferably comprises 12.5 to 40 wt % lipid, more preferably 19 to 30 wt %. Preferably the lipid comprises the essential fatty acids alpha-linolenic acid (ALA), linoleic acid (LA) and/or long chain polyunsaturated fatty acids (LC-PUFA). The LC-PUFA, LA and/or ALA may be provided as free fatty acids, in triglyceride form, in diglyceride form, in monoglyceride form, in phospholipid form, or as a mixture of one of more of the above. Preferably the present nutritional composition comprises at least one, preferably at least two lipid sources selected from the group consisting of rape seed oil (such as colza oil, low erucic acid rape seed oil and canola oil), high oleic sunflower oil, high oleic safflower oil, olive oil, marine oils, microbial oils, coconut oil, palm kernel oil. The present nutritional composition is not human milk.

The present nutritional composition preferably comprises protein. The protein used in the nutritional composition is preferably selected from the group consisting of non-human animal proteins, preferably milk proteins, vegetable proteins, such as preferably soy protein and/or rice protein, and mixtures thereof. The present nutritional composition preferably contains casein, and/or whey protein, more preferably bovine whey proteins and/or bovine casein. Thus in one embodiment the protein in the present nutritional composition comprises protein selected from the group consisting of whey protein and casein, preferably whey protein and casein, preferably the whey protein and/or casein is from cow's milk. Preferably the protein comprises less than 5 wt % based on total protein of free amino acids, dipeptides, tripeptides or hydrolysed protein. The present nutritional composition preferably comprises casein and whey proteins in a weight ratio casein:whey protein of 10:90 to 90:10, more preferably 20:80 to 80:20, even more preferably 35:65 to 55:45.

The wt % protein based on dry weight of the present nutritional composition is calculated according to the Kjeldahl-method by measuring total nitrogen and using a conversion factor of 6.38 in case of casein, or a conversion factor of 6.25 for other proteins than casein. The term ‘protein’ or ‘protein component’ as used in the present invention refers to the sum of proteins, peptides and free amino acids.

The present nutritional composition preferably comprises protein providing 1.6 to 4.0 g protein per 100 kcal of the nutritional composition, preferably providing 1.6 to 3.5 g, even more preferably 1.75 to 2.5 g per 100 kcal of the nutritional composition. In one embodiment, the present nutritional composition comprises protein providing 1.6 to 2.1 g protein per 100 kcal of the nutritional composition, preferably providing 1.6 to 2.0 g, more preferably 1.7 to 2.1 g, even more preferably 1.75 to 2.0 g per 100 kcal of the nutritional composition. In one embodiment, the present nutritional composition comprises protein in an amount of less than 2.0 g per 100 kcal, preferably providing 1.6 to 1.9 g, even more preferably 1.75 to 1.85 g per 100 kcal of the nutritional composition. A too low protein content based on total calories will result is less adequate growth and development in infants and young children. A too high amount will put a metabolic burden, e.g. on the kidneys of infants and young children. When in liquid form, e.g. as a ready-to-feed liquid, the nutritional composition preferably comprises 0.5 to 6.0 g, more preferably 1.0 to 3.0 g, even more preferably 1.0 to 1.5 g protein per 100 ml, most preferably 1.0 to 1.3 g protein per 100 ml. Based on dry weight the present nutritional composition preferably comprises 5 to 20 wt % protein, preferably at least 8 wt % protein based on dry weight of the total nutritional composition, more preferably 8 to 14 wt %, even more preferably 8 to 9.5 wt % protein based on dry weight of the total nutritional composition.

In order to meet the caloric requirements of an infant, the nutritional composition preferably comprises 45 to 200 kcal/100 ml liquid. For infants the nutritional composition has more preferably 60 to 90 kcal/100 ml liquid, even more preferably 65 to 75 kcal/100 ml liquid. This caloric density ensures an optimal ratio between water and calorie consumption. The osmolarity of the present composition is preferably between 150 and 420 mOsmol/l, more preferably 260 to 320 mOsmol/l. The low osmolarity aims to further reduce the gastrointestinal stress, which may affect sleep.

When the nutritional composition is in a ready to feed, liquid form, the preferred volume administered on a daily basis is in the range of about 80 to 2500 ml, more preferably about 200 to 1200 ml per day. Preferably, the number of feedings per day is between 1 and 10, preferably between 3 and 8. In one embodiment the nutritional composition is administered daily for a period of at least 2 days, preferably for a period of at least 4 weeks, preferably for a period of at least 8 weeks, more preferably for a period of at 25 least 12 weeks, in a liquid form wherein the total volume administered daily is between 200 ml and 1200 ml and wherein the number of feedings per day is between 1 and 10.

The present nutritional composition, when in liquid form, preferably has a viscosity between 1 and 60 mPa·s, preferably between 1 and 20 mPa·s, more preferably between 1 and 10 mPa·s, most preferably between 1 and 6 mPa·s. The low viscosity ensures a proper administration of the liquid, e.g. a proper passage through the whole of a nipple. Also this viscosity closely resembles the viscosity of human milk. Furthermore, a low viscosity results in a normal gastric emptying and a better energy intake, which is essential for infants which need the energy for optimal growth and development. The present nutritional composition alternatively is in powder form, suitable for reconstitution with water to a ready to drink liquid. The present nutritional composition is preferably prepared by admixing a powdered composition with water. Normally infant formula is prepared in such a way. The present invention thus also relates to a packaged power composition wherein said package is provided with instructions to admix the powder with a suitable amount of liquid, thereby resulting in a liquid composition with a viscosity between 1 and 60 mPa·s. The viscosity of the liquid is determined at a shear rate of 95 s⁻¹ at 20° C.A suitable equipment to measure the viscosity is Physica Rheometer MCR 300 (Physica Messtechnik GmbH, Ostfilden, Germany).

### Application

The methods or uses according to the present invention comprising administering the present nutritional composition also refer to administering an effective amount of the nutritional composition to the subject in need thereof. The methods or uses according to the present invention are considered to be non-therapeutic methods or uses.

Sleep duration is defined as the total length of sleep in h per day (a 24 h period). An improved sleep efficiency is defined as spending a larger period of time asleep between sleep onset and wakefulness. Sleep episode duration, sometimes referred to as nap time, is the length of one sleep episode. Sleep frequency is defined as the number of sleep episodes per day (24 h). Wake episodes is defined as the number of wake episodes per day (24 h). Sleep pattern is defined as the pattern of sleep and wake episodes in frequency and duration during the day (24 h). Sleep behaviour is defined as the sleep pattern and sleep duration during the day (24 h). Maturation of sleep or development of sleep pattern in infants is the maturation of sleep or development of the sleep pattern towards a decreased sleep duration, a decreased sleep and wake frequency and an increase sleep efficiency over time, preferably over the first year of life, preferably the first 4 months (17 weeks) of life.

Sleep pattern and sleep behavior develops rapidly during the first few years of life and is a highly dynamic process. While the need for day sleep decreases, night sleep duration increases through the first year of life, resulting in a shift towards more nocturnal patterns of sleep. Researchers found that greater sleep efficiency (i.e. spending a larger percentage of time asleep between sleep onset and wakefulness) was positively correlated with scores on the Bayley Scales of Infant and Toddler Development second edition (BSID-II) Mental Development Index (MDI) amongst 10-month-old infants. Researchers found that 11- to 13-month-old infants who had greater sleep efficiency measured via sleep actigraphy data also displayed better overall cognitive problem-solving skills as measured by the Ages and Stages Questionnaire. Alterations in sleep organization early in life may persist into childhood: a study showed, for example, lower sleep efficiency and more awakenings during the sleep period in IUGR children aged 4-7 years.

The inventors found an improvement in the development of the sleep pattern or sleep behavior in infants that were administered a nutritional composition with partly fermented composition and non-digestible oligosaccharides. In particular above 13 weeks an increase in sleep efficiency and decrease in sleep frequency was observed. This was not related to symptoms of colics or crying behavior, as the peak for this was observed at an earlier age, between 4 and 7 weeks (data not shown). A decreased sleep frequency and increased sleep efficiency or increased duration of a sleep episode is especially beneficial if it occurs above 13 weeks. Before that time infants should feed more regularly because of the small size of the stomach of newborns and hence sleep episode should not be too long. However above 13 weeks of age the infant's stomach is large enough to consume a sufficient volume to sustain longer periods of sleep.

Therefore, in one embodiment the current invention relates to a method or use for improving sleep pattern and/or improving sleep behavior in an infant. This is achieved by administering a nutritional composition that is at least partly fermented by lactic acid producing bacteria and comprises non-digestible oligosaccharides as described above. Preferably improving sleep efficiency, decreasing sleep frequency, decreasing wake frequency and/or increasing sleep episode duration in an infant is established above 3 months (13 weeks) of age.

In one embodiment the current invention relates to a method or use for improving the development of sleep pattern and/or improving maturation of sleep pattern in an infant. This is achieved by administering a nutritional composition that is at least partly fermented by lactic acid producing bacteria and comprises non-digestible oligosaccharides as described above. Preferably development of sleep pattern and/or maturation of sleep pattern occurs in the infant under 3 months of age.

In one embodiment the current invention relates to a method or use for improving sleep efficiency, decreasing sleep frequency, decreasing wake frequency and/or increasing sleep episode duration in an infant. This is achieved by administering a nutritional composition that is at least partly fermented by lactic acid producing bacteria and comprises non-digestible oligosaccharides as described above. Preferably improving sleep efficiency, decreasing sleep frequency, decreasing wake frequency and/or increasing sleep episode duration in an infant is established above 3 months (13 weeks) of age.

For all the methods and uses the claimed effects on sleep (improvements, decreasing frequency and increasing sleep episode duration) are when compared to infants not being administered the nutritional composition of the present of invention; in other words infants being administered a nutritional composition that is not at least partly fermented and does not comprise non-digestible oligosaccharides.

For all the methods and uses the claimed effects on sleep (improvements, decreasing frequency and increasing sleep episode duration) preferably occur when the infant is above 3 months of age (above 13 weeks). In the context of the present invention, 3 months is the same as 13 weeks.

The present nutritional composition is administered to an infant, i.e. a human subject with an age of 0 to 12 months, more preferably in an infant with an age of 0 to 6 months, most preferably an infant of 0 to 4 months. Preferably the nutritional composition of the present invention is starting to be administered to the infant when the infant is below 3 months of age. Preferably the nutritional composition is administered for at least 1 week, more preferably for at least 4 weeks, more preferably for at least 8 weeks, more preferably for at least 1 week during the first 3 months of life, more preferably for at least 4 weeks during the first 3 months of life, more preferably for at least 8 weeks during the first 3 months of life, more preferably during the first 3 months of life. In a preferred embodiment, the methods or uses according to the present invention are for healthy infants, preferably for healthy, term infants.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one. Wt % means weight percentage. Unless mentioned otherwise a day refers to a period of 24 h (starting and ending at midnight).

### Example: Double-Blind, Randomized, Controlled Trial in Healthy, Term Infants

### Participants

Parents and their infants were recruited from paediatric medical clinics in Italy (3 sites) and Spain (6 sites). Only parents who autonomously decided to exclusively formula feed their infant were informed of the study. Eligible infants were term-born 37 and 42 weeks gestational age), of normal birth weight (10^{th} to 90^{th} percentile according to applicable growth charts), ≤28 days of age and having a head circumference within +/−2SD according to WHO growth standards. Infants with a known increased risk of cows' milk allergy, soy allergy, lactose intolerance, any medical condition that could interfere with study outcomes or having a mother suffering from (gestational) diabetes were excluded from participation. Infants meeting all criteria but fed with an infant formula (IF) containing probiotics or synbiotics prior to study entry were also excluded from participation. Written informed consent was obtained from all parent(s) or guardian(s) before enrolment to the study.

### Trial Design

This study was a multi-centre, prospective, double-blind, randomised control trial designed to explore the incidence of GI symptoms, stool characteristics, growth adequacy and safety in healthy, term-born infants up to 17 weeks of age. Upon enrolment, exclusively IF fed infants were assigned to one of two formulae using a computer-generated randomisation number with country, centre and sex as strata. Both the investigators and the infants' parents were blinded to the formulae and the randomisation details. Inclusion of twins was allowed and were to be randomized to the same product group. An interactive web response system was used by the investigator to provide each subject with their unique study number when enrolled. During the study infants were fully formula-fed; only use of water, tea or rehydration solutions, drops or syrups (vitamins, minerals, medicines, but not probiotics) was allowed. This study was conducted according to the guidelines laid down in the Declaration of Helsinki and all procedures were reviewed and approved by the relevant Ethical Committees in participating countries.

### Study Product

The intervention formulas were comparable in nutritional composition; cow's milk based, iso-caloric (66 kcal/100 ml) products containing similar amounts of protein (1.2 g/100 ml; whey protein/casein wt/wt 1/1), lipids (3.4 g/100 ml; mainly vegetable oil), 7.7 g digestible carbohydrates (mainly lactose) vitamins and minerals, manufactured per good manufacturing practices (ISO 22000) and compliant with Directive 2006/141/EC.

The experimental infant formula contained the specific mixture of non-digestible oligosaccharides (0.8 g/100 ml) prebiotic mixture scGOS/IcFOS (9:1 wt/wt) and contained fermented formula in a proportion of 30 wt % of the total composition based on dry weight. The fermented formula fraction underwent a unique fermentation process (Lactofidus™) with two bacterial strains Bifidobacterium breve C50 and Streptococcus thermophilus 065. The infant formula comprised about 0.33 wt % of the sum of lactic acid and lactate based on dry weight of the composition, of which at least 95 wt % was L-lactic acid+L-lactate. As a source of scGOS Vivinal® GOS (Friesland Campina DOMO) was used and as a source of IcFOS Raftiline HP® (Orafti) was used.

The control formula did not contain non-digestible oligosaccharides and no fermentation process was applied. Both products had a similar taste, smell, and appearance.

### Measurements

The exploratory outcomes included gastrointestinal symptoms as well as measures of infant growth, stool characteristics, formula intake and adverse events, sleep and crying episodes and duration. The baseline visit occurred ≤28 days of age and infants were assessed at 4, 8, 13, and 17 weeks of age thereafter. Demographic information and infant characteristics were collected by interview at the baseline visit.

At each study visit, infant anthropometrics were measured; the weight for each infant was registered by weighing them naked, on calibrated electronic scales, supine length of infants was registered by using a standard measuring board and a non-stretchable slotted insertion tape was used to measure head circumference. Adverse events and the use of concomitant medication, drinks and food were documented by the investigators at each visit. For adverse events the start and stop date, severity and taken actions were documented. Moreover, the investigators documented the probability of any relationship with the study product.

Daily diaries were filled in by the parents during the entire study duration (up to 17 weeks of age) and recorded stool frequency and consistency as well as crying and sleeping behavior. Crying and sleeping behavior was recorded using a modified Baby day diary with a 24 h bar to document crying and sleeping episodes (Vandenplas, Y., et al., Acta Paediatr, 2017. 106(7): p. 1150-1158).

In the 7-d period preceding each visit, parents recorded study formula intake and the occurrence and severity of gastrointestinal symptoms (e.g. regurgitation, flatulence, abdominal distension) based on a 4-point scale (absent/mild/moderate/severe). At each visit, the completion of the diaries was discussed with the parents and verified for its completion and plausibility by the investigator. In addition to the parent's perceived and recorded GI symptoms, incidences of functional gastrointestinal disorders were also evaluated applying adapted Rome III criteria to the daily diary recordings. In addition to the study visits, a total of three telephone calls were conducted between assessment visits to discuss parental questions, record any illness or medications and to monitor protocol compliance.

### Statistics

For all diary data, a daily average or a daily total was calculated for those parameters where more than one entry per day was possible (e.g. GI symptoms, stool consistency, sleep and crying duration). All diary data was assigned to specified windows corresponding to the study visits and/or weeks of age. The derived parameters were only calculated if records included at least 3 days of data per week. The specified windows were 14-42 days of age for visit 2, 43-73 days of age for visit 3, 74-104 days of age for visit 4, and 105-133 days of age for visit 5. For week of age, the diary information was assigned to the period of ±3 days the exact days of age (e.g. 4 weeks=25-31 days).

For comparison of the intervention groups with the WHO Child Growth, an analysis of growth parameter z-scores using WHO growth trajectories were performed by using a mixed model with adjustment of baseline z-score.

Apart from the growth equivalence analysis, all parameters of the two intervention groups were compared using a two-sample t-test or Wilcoxon rank-sum test for continuous data, and the chi-square test or Fisher's exact test for categorical data, as appropriate. Equivalence analyses for weight gain, length gain and head circumference gain were performed using Parametric Curves Mixed model (PC) which describes the development of growth parameters over time by a second order polynomial curve, with the stratification factors as a fixed effect, and each subject's intercept and slope as random effects. Equivalence between intervention groups was demonstrated when the two-sided 90% Cl of the difference in means in daily gain laid within the pre-defined −0.5SD to +0.5SD equivalence margins. The data analysis was conducted with SAS software (SAS Institute Inc., Cary, N.C., version 9.4 for Windows). Unless stated otherwise, the per protocol analysis is presented. In the per protocol analyses, eligibility of data was assessed on visit level. In the per protocol growth outcomes analysis (PP-G) data of subjects that met the inclusion criteria, were protocol compliant, and had at least one post-baseline visit with anthropometric data collection was included. In addition, apart from protocol compliance, the per protocol analysis of tolerance and several other outcomes required availability of diary data and is referred to as the per protocol tolerance (PP-T) population.

### Results

A total of 200 infants were randomised in this trial. A total of 152 infants completed the study of which 72 and 80 infants were part of the experimental and control groups, respectively, resulting in a drop-out rate of 21%. The number and reasons for early termination were not different between intervention groups and included: no longer wished to participate in the trial (n=44), subjects with an AE (n=14), loss to follow up (n=13), and moved out of the region (n=2). Of the total study population, 5 infants were excluded from all per protocol analyses due to major protocol violations including no study product taken (n=2), cows' milk allergy (n=1), failure to thrive (n=1), and unknown last intake data (n=1). Three sets of twins were also excluded (n=6) from the per protocol analyses as they were accidentally provided with differing products. Additional subjects were excluded from the PP-T population due to lack of any diary data (n=36) and/or introduction of non-study formula (n=8) and from the PP-G due to lack of post-baseline visit (n=47), introduction of non-study formula (n=12), not meeting birth weight criteria (n=9), gestational diabetes (n=1), delayed start of study product intake (n=1) or use of glucocorticoids (n=1). Demographic data were not apparently different between the intervention groups for the ITT population as well as both PP populations (data not shown).

### Study Product Intake

Infants in both intervention groups consumed an increasing amount of formula during the study period. No significant differences were shown at any timepoint up to 17 weeks of age for volume intake or number of feedings per day between the experimental and control groups in the ITT, PP-G or PP-T populations (data not shown).

### Gastrointestinal Symptoms

The overall parent-reported incidence of GI and related symptoms (constipation, diarrhoea, flatulence, abdominal distension, regurgitation, vomiting, diaper dermatitis, and arching of the back) with a score of moderate or severe at least once in the study period was not different between intervention groups, with an incidence of 85.7% in the experimental group and 86.0% in the control group. In addition, no significant differences between the formula groups were observed in the specific incidences of any of the reported GI symptoms during the study (P>0.1).

Interestingly, the total incidence of gastrointestinal disorders reported by the investigators as adverse events was only 14-18%. No relevant differences in parent- or investigator-reported incidence or severity of GI symptoms were observed between both formula groups, apart from a significantly lower incidence of infantile colic reported as an adverse event in the experimental group. To conclude, both infant formulae either or not containing fermented formula (and its affiliated postbiotics) and prebiotics are well-tolerated.

Colics is known to be at its peak in infants of 2 months, and after 8 weeks rapidly declines, and at 3 months of age most cases of colics will have subsided. Indeed a decrease in infant crying from the second month of life onward was observed. The peak in total crying duration of 1.3 hours per 24 hours, observed in the study presented here in week 4-7, is in line with the previously reported values of 1.6 hours per 24 hours.

Stool consistency was softer in the experimental vs control group with values closer to the breastfed reference group. Equivalence in daily weight gain in both formula groups was demonstrated with growth outcomes close to those of breastfed infants and WHO growth standards. No clinically relevant differences in number, severity, relatedness or type of (serious) adverse events were observed,

### Effects on Sleep

At baseline the reported sleep duration and sleep frequency was not significantly different between the groups. In general, the median number of parent-reported sleep episodes decreased in all groups over the 17-week study period (Table 1). This is in line with a normal development of sleep in infants. No significant differences in the number of reported sleep episodes were observed between the formula groups until after 13 weeks of age. In week 14 and thereafter a consistent and significant lower median number of sleep episodes was observed in the experimental group (5.5-6.1 n/d) vs control group (6.2-6.7/d) (P<0.07).

Parent-reported sleep duration decreased over the intervention period in all groups, with a range of medians from 13.9-20.0 h/d for the experimental group, 13.9-20.0 h/d for the control group (Table). The total sleep duration and decrease over time are in line with normal sleep duration and sleep duration development in infants.

TABLE
Summary of sleeping frequency (episodes/day) and sleeping duration (hours/day) per week of age.

| | | Experimental Group | | Control Group | |
|---|---|---|---|---|---|
| | | No of sleep | Duration of sleep | No of sleep | Duration of sleep |
| | | episodes/24 h | (h/24 h) | episodes/24 h | (h/24 h) |
| Age | | N = 77 | | N = 86 | |
| 4 wks | n | 55 | | 61 | |
| | Median (Q1-Q3) | 7.2 (6.3-8.5) | 15.0 (12.5-16.7) | 7.3 (6.0-8.0) | 15.0 (13.5-16.3) |
| 5 wks | n | 67 | | 74 | |
| | Median (Q1-Q3) | 7.5 (6.3-8.1) | 14.4 (12.7-15.5)** | 7.2 (6.7-8.3) | 15.3 (14.0-16.1) |
| 6 wks | n | 66 | | 77 | |
| | Median (Q1-Q3) | 7.0 (6.0-8.1) | 14.1 (12.8-16.0) | 7.3 (6.6-8.0) | 14.9 (13.5-16.0) |
| 7 wks | n | 67 | | 75 | |
| | Median (Q1-Q3) | 7.1 (6.1-8.3) | 14.2 (12.6-15.9) | 7.0 (6.1-7.7) | 14.8 (12.7-15.6) |
| 8 wks | n | 65 | | 76 | |
| | Median (Q1-Q3) | 6.7 (5.3-7.8) | 14.1 (13.1-15.9) | 6.9 (6.3-7.8) | 14.5 (13.4-16.3) |
| 9 wks | n | 66 | | 76 | |
| | Median (Q1-Q3) | 6.7 (5.6-8.2) | 14.4 (13.2-15.6) | 7.0 (6.3-7.6) | 14.7 (13.1-15.8) |
| 10 wks | n | 64 | | 75 | |
| | Median (Q1-Q3) | 6.6 (5.5-7.9) | 14.1 (13.0-15.5) | 6.7 (5.9-7.6) | 14.2 (13.0-15.6) |
| 11 wks | n | 64 | | 75 | |
| | Median (Q1-Q3) | 6.7 (5.5-7.6) | 14.1 (12.8-15.6) | 6.7 (6.0-7.3) | 14.2 (12.7-15.6) |
| 12 wks | n | 64 | | 75 | |
| | Median (Q1-Q3) | 6.5 (5.6-7.6) | 14.0 (13.1-15.3) | 6.7 (6.0-7.4) | 14.2 (12.8-15.6) |
| 13 wks | n | 63 | | 74 | |
| | Median (Q1-Q3) | 6.5 (5.4-7.5) | 14.2 (13.2-15.3) | 6.5 (5.7-7.3) | 14.1 (12.9-15.1) |
| 14 wks | n | 64 | | 70 | |
| | Median (Q1-Q3) | 6.1 (5.1-6.9)** | 14.2 (13.3-15.2) | 6.7 (5.7-7.4) | 14.0 (12.9-14.5) |
| 15 wks | n | 63 | | 72 | |
| | Median (Q1-Q3) | 6.1 (5.0-6.9)* | 13.7 (12.9-14.9) | 6.4 (5.6-7.1) | 14.1 (12.9-15.0) |
| 16 wks | n | 62 | | 72 | |
| | Median (Q1-Q3) | 5.8 (5.0-7.1)* | 13.8 (13.0-14.8) | 6.4 (5.6-7.2) | 13.8 (12.7-14.8) |
| 17 wks | n | 29 | | 40 | |
| | Median (Q1-Q3) | 5.5 (5.0-6.3)** | 13.9 (13.0-15.3) | 6.2 (5.7-6.8) | 13.9 (13.2-14.7) |

*<0.1 COMPARED TO CONTROL GROUP
**P < 0.05 COMPARED TO CONTROL GROUP

The number of episodes of sleep per 24 h in infants over 13 weeks of age was over 24 h was consistently decreased, and concomitantly also the episodes of wake. At the same time the total duration of sleep and wake however, was not different between the two groups. Based on this, the duration per sleep episode was calculated, and found that this is increased in infants above 13 weeks in the experimental group. The average difference per sleep episode at the end of the study was around 12-15 minutes, with the longer sleep episode being observed in the experimental group.

Vandenplas et al (2017 Acta Paediatrica 106, pp. 1150-1158) tested a partly fermented formula with non-digestible oligosaccharides and found no indication of a statistically significant difference in the number of sleeping episodes or sleeping duration at any time point for any study group comparison. In this study a partly fermented formula comprising non-digestible oligosaccharides was compared with non-fermented formula comprising non-digestible oligosaccharides, and partly fermented formula without non-digestible oligosaccharides. There was no comparison with a formula that was not partly fermented and did not comprise non-digestible oligosaccharides which explains why the study is inconclusive with regard to determining whether there is a statistically significant effect on sleeping episodes or sleeping duration of both fermented formula and non-digestible oligosaccharides. Moreover the study does not measure on a weekly basis, which lowers the statistical sensitivity of the study. The results of the present trial thus support the finding that both the fermented part and the non-digestible oligosaccharides need to be present in order to achieve an effect on sleep.

The results found in the clinical trial are indicative that upon administration of a nutritional composition that is at least partly fermented by lactic acid producing bacteria and comprises non-digestible oligosaccharides to infants an improvement of sleep behavior and/or improvement of sleep pattern is obtained. Moreover, the results are indicative that an improvement of sleep efficiency, a decrease of sleep frequency, a decrease of wake frequency or an increase of sleep episode duration is obtained. Also the results are indicative that an improvement of the development of sleep pattern or improvement of maturation of sleep pattern in the infant is obtained.

1. A method for improving sleep behavior and/or sleep pattern in an infant, comprising administering to the infant a nutritional composition that is at least partly fermented by lactic acid producing bacteria and comprises non-digestible oligosaccharides to the infant, wherein the nutritional composition comprises 0.1 to 1.5 wt % of the sum of lactic acid and lactate based on dry weight of the composition, and wherein the composition comprises 2.5 to 15 wt % non-digestible oligosaccharides based on dry weight of the nutritional composition and the non-digestible oligosaccharides are selected from the group consisting of galacto-oligosaccharides and fructo-oligosaccharides, and wherein the nutritional composition is an infant formula or a follow on formula, wherein the sleep behavior and/or sleep pattern is improved compared to infants being administered an infant formula or a follow on formula that is not at least partly fermented and does not comprise non-digestible oligosaccharides, wherein the nutritional composition is administered to the infant for at least 4 weeks.

2. The method according to claim 1 wherein improving sleep behavior and/or improving sleep pattern comprises improving sleep efficiency, decreasing sleep frequency, decreasing wake frequency and/or increasing sleep episode duration in an infant above 3 months of age.

3. The method according to claim 1, wherein improving sleep behavior and/or improving sleep pattern comprises improving the development of sleep pattern and/or improving maturation of sleep pattern in the infant.

4. The method according to claim 1, wherein the infant is a healthy, term infant.

5. The method according to claim 1, wherein the sum of L-lactic acid and L-lactate is more than 50 wt % based on the sum of total lactic acid and lactate.

6. The method according to claim 1, wherein the composition comprises lactic acid producing bacteria.

7. The method according to claim 6, wherein the lactic acid producing bacteria are selected from the group consisting of Bifidobacterium and Streptococcus.

8. The method according to claim 6 or 7, wherein the lactic acid producing bacteria are selected from the group consisting of Bifidobacterium breve and Streptococcus thermophilus.

9. The method according to claim 1, wherein the nutritional composition comprises 3.0 to 10 wt % non-digestible oligosaccharides based on dry weight of the nutritional composition.

10. The method according to claim 1, wherein the nutritional composition comprises 1.7 to 2.1 g protein per 100 kcal.

11.-13. (canceled)

14. The method according to claim 7, wherein the lactic acid producing bacteria are Bifidobacterium and Streptococcus.

15. The method according to claim 8, wherein the lactic acid producing bacteria are Bifidobacterium breve and Streptococcus thermophiles.

16. The method according claim 10, wherein the nutritional composition comprises 1.75 to 2.0 g protein per 100 kcal.
